(19) United States
(12) Patent Application Publication
(10) Pub. No.: US 2024/0278036 A1
(43) Pub. Date: Aug. 22, 2024

(54) Multifrequency Neurostimulation Device For Improving Cognitive Performance Of Brain

(71) Applicant: Revo Dungca, Scarborough (CA)

(72) Inventor: Revo Dungca, Scarborough (CA)

(21) Appl. No.: 18/581,103

(22) Filed: Feb. 19, 2024

Related U.S. Application Data
(60) Provisional application No. 63/486,014, filed on Feb. 20, 2023.
(60) Provisional application No. 63/521,565, filed on Jun. 16, 2023.

(51) Int. Cl. A61N 5/06

(52) U.S. Cl. CPC A61N 5/0618; A61N 2005/0626; A61N 2005/0632; A61N 2005/0652; A61N 2005/0659; A61N 2005/0663

(57) ABSTRACT

A multifrequency neurostimulation device for improving cognitive performance of brain is disclosed. The device includes a portable brain entrainment device, comprising a body, a plurality of buttons enable plurality of operations, a light flicker module for emitting varying range of light frequency, a light intensity adjustment knob, a sound module for generating varying range of sound frequency, a volume adjustment knob, a control unit to synchronize the operation of the light flicker module and sound module as per a user's command, a timer tracks time duration of a light and/or sound therapy, a plurality of time-indicating LED (Light emitting diodes) lights indicates remaining time, a rechargeable battery for supplying power, and a USB (Universal Serial Bus) port enables establishment of a wired connection between the device and an external power source via a USB with an inbuilt voltage converter for recharging the rechargeable battery.

### CROSS-REFERENCE TO RELATED PATENT APPLICATION

This disclosure claims the benefit of the priority of U.S. Provisional Patent Application No. 63/486,014, entitled “A Portable Brain Entrainment Device” and filed on Feb. 20, 2023; Patent Application No. 63/521,565, entitled “A Multi-Functional Light Flicker Device” and filed on Jun. 16, 2023. The above-identified applications are incorporated herein by reference in its entirety.

### FIELD OF THE INVENTION

The present invention relates neurostimulation technologies and more particularly relates to multifrequency neurostimulation device for improving cognitive performance of brain capable of delivering specific audiovisual stimuli to induce desired cognitive states in a user, in a convenient and effective manner by offering a compact structure and user-friendly interface.

### BACKGROUND OF THE INVENTION

Brain entrainment, brainwave entrainment/synchronization or neural entrainment is the synchronization of the neural oscillations with external stimuli such as lights, touch and sound. This has been observed that the brainwaves naturally synchronize to the rhythm of periodic external stimuli. For example: some researchers have observed that the acoustic entrainment of delta waves in slow wave sleep had the functional effect on improving memory in healthy users. Brainwave entrainment has been recognized for its potential to modulate cognitive states and improve well-being by reducing anxiety and stress. Improved well-being corresponds to improved focus, concentration, memory, mood boost, enhanced meditation, reduced stress and anxiety. The brainwave entrainment equipment that is currently available in the market is complex and stationary in structure.

Currently, the available brain entrainment utilizes different approaches such as magnetic, electric, haptic or vibrational and audio stimulation for creating artificial rhythms. There are few devices that utilize electric impulse to reduce stress and improve sleep. However, these devices are intended to be worn on forehead and back of the neck, and uses an adhesive strips that needs to be pasted on the forehead, which is uncomfortable and also wear out quickly. Similarly, one of the device utilizes vibration to send signals from wrist or ankle up through the nerve or/and into the brain to help with focus, sleep, calm and physical recovery. The device needs to worn as a bracelet around wrist or ankle, which is again uncomfortable.

In one of the prior arts U.S. Pat. No. 8,968,176B2 discloses a sound and light machine (SLM) produces sound and light pulses at brain wave frequencies so as to induce a particular state of consciousness in a user, while simultaneously providing music. The SLM includes light emitting diodes (LEDs) and headphone speakers. A microcontroller runs firmware to control the LEDs and headphone speakers so as to produce a plurality of dominant brainwave frequencies. Binaural voices produce a melody of music without interfering with the brainwave frequencies. Each note of the melody is sounded at a specific pitch based on information in a Tone Object, which includes a state variable index holding the note's current position within a waveform array and a state variable step, which is an amount by which to increment the state variable index at each iteration. The index is set to 0 at startup and the step is calculated from the initial note desired. Although, this particular prior art discloses the portable brain entrainment device that utilizes light and sound. However, the above-mentioned prior art discloses a guide glass, which lacks a compact structure and user-friendly interface, which is uncomfortable and does not allow the user to customize time of therapy and frequency to obtain desired cognitive state. Thus, the cited prior art does not serve the purpose of eliminating the aforementioned drawbacks.

In another relevant art U.S. Ser. No. 11/172,859B2 relates to wearable brain activity device with electrodes held on a person's head by a frame comprising a ring portion which encircles the person's head and an arc portion which loops over the top of the person's head. Data from the electrodes is used to analyze the person's brain activity within a frequency band of 0.5-4 Hz. The device includes a speaker or vibrating member which is used to help to guide the person's brain activity from a first pattern to a second pattern. Although, the mentioned art provides the portable brain entrainment device that utilizes different approaches for brain entrainment such as vibration, light and sound. However, the device needs to be plugged-in while performing operation and also does not offer portable features.

Conventionally, many tools and devices are available in the market that is capable of being used for brain entrainment. However, these tools and devices lack a compact structure and user-friendly interface, which is uncomfortable and does not track time. Moreover, these tools and devices does not provide a means to change the frequency of sound and light intensity to select a range of pre-programmed sessions designed to promote relaxation, focus, sleep or other cognitive states.

To overcome the above-mentioned shortcoming, a portable brain entrainment device is being introduced that is capable of delivering a wide-range of audiovisual stimuli to induce desired cognitive states in a user, in a convenient and effective manner by offering a compact structure and user-friendly interface.

### OBJECTS OF THE INVENTION

The principal object of the present invention is to overcome the disadvantages of the prior art.

The object of this invention is to provide a multifrequency neurostimulation device for enhanced beta and gamma brainwave activity. Another object of the invention is provide a portable brain entrainment device that enables a user to select a wide-range of audiovisual stimuli to improve cognitive performance of brain, which promote relaxation, focus, sleep or other cognitive states. Thus provide a tailored experience for each user.

Yet another object of the invention is to provide an intersection of neuroscience and neurotechnology focusing on neurostimulation devices engineered to specifically modulate brainwave frequencies for therapeutic benefits and cognitive enhancement.

Yet another object of the present invention is to provide a device that is portable in nature for offering advantage of accessibility, allowing user(s) to access cognitive state anywhere, anytime.

Another object of the present invention is to provide a device that helps the user(s) to manage his/her time by tracking time invested in brainwave entrainment, without the need for specialized equipment or facilities.

Another object of the present invention is to provide a device that increases beta brainwave activity to improve alertness, concentration, and cognitive functioning.

Another object of the present invention is to provide a device that enhances gamma brainwave activity for superior information processing, memory recall, and neural synchronization.

Another object of the present invention is to provide a device that offers a means of versatile neuro-stimulation for research and therapeutic applications, allowing for personalized cognitive and mental health interventions.

Yet another object of the invention is to provide a compact structure and user-friendly interface with re-charging facility.

The foregoing and other objects, features, and advantages of the present invention will become readily apparent upon further review of the following detailed description of the preferred embodiment as illustrated in the accompanying drawings.

### SUMMARY OF THE INVENTION

This present invention relates to a multifrequency neurostimulation device for improving cognitive performance of brain capable of delivering specific audiovisual stimuli to induce desired cognitive states in a user. The multifrequency neurostimulation device includes a portable brain entrainment device and or light flicker device that provides a means to optimize cognitive performance and enhance mental well-being. Harnessing the power of synchronized audiovisual stimuli, the present invention offers user(s) a convenient and effective means of achieving desired brainwave state such as relaxed, focus and improved sleep. The device is aided with a compact and sleek structure, intuitive interface and portable functionality. Moreover, the present invention empowers individuals to unlock their full potential anytime, anywhere.

According to an embodiment of the present invention, a multifrequency neurostimulation device for improving cognitive performance of brain comprises of a body having a top portion and bottom portion configured to house a plurality of components, which includes a plurality of buttons for enabling a user to generate a command for plurality of operations such as switching ON/OFF the device, play, pause, reverse, forward and color change or temperature; a light emitting module configured to emit a varying range of light frequency; a light adjustment knob configured for allowing the user to customize light frequencies required for a desired cognitive state; a sound module configured to generate a varying range of sound frequency; a volume adjustment knob configured for allowing the user to customize sound frequencies required for the desired cognitive state; a microcontroller to control the operation of the light emitting module and the sound module; a timer configured for tracking time duration of emitted lights and/or sound; a plurality of time-indicating LED (Light emitting diodes) lights indicates remaining time duration; and a rechargeable battery configured on the body for supplying power to electrical and electronic component associated with the device.

In an embodiment of the present invention, the proposed device is configured to provide the varying range of light and sound frequency, which ranges from 20-45 Hertz (Hz). The varying range of frequency is a pre-defined frequency range. In alternate embodiment of the present invention, the device is configured to provide frequency 20 Hz, 40 Hz, and 20-40 Hz in random order.

According to a preferred embodiment of the present invention, a portable brain entrainment device for stimulating gamma brain wave patterns to improve brain health comprises of a body configured to encase a plurality of components, the components includes; a speaker to generate an audible binaural beats and monotone sound frequencies; a volume adjustment knob configured to adjust the volume of the speaker; a light emitter comprising a plurality of LED's for producing light flickering/flashing effect; a light intensity adjustment knob configured to adjust the brightness of the light from the light emitter; a timer configured for effectively managing and tracking amount of time a user needs on a task to improve productivity and time efficiency; a power supply unit to supply power to the plurality of components, wherein the power supply unit comprises a rechargeable battery to provide mobility to the device; and a USB port with built-in converters allowing the user to connect an external power source to recharge a power supply unit.

In an embodiment of the present invention, the speaker and the light emitter produce an audible sound and light flickering/flashing effect of 40 Hz frequency simultaneously; wherein the light emitter is configured to a customizable lighting experience to a variety of uses via an ability to change the color and temperature.

In the embodiment, the light emitter emits a plurality of color of light to enhance ergonomic usage of the device in day and evening without hindering circadian sleep cycle. In the embodiment, the light emitter has a 50,000 lifespan and a full spectrum of at least RA90. In the embodiment, the light intensity adjustment knob allows adjustment of brightness of light at varying percentage such as 25%, 50%, and 100%.

According to another embodiment of the present invention, a portable multi-functional light flicker device for stimulating beta brain waves to improve cognitive performance of the brain is disclosed, device comprises of a body having a top portion and bottom portion configured to hold a plurality of components of the device; a plurality of LEDs (Light Emitting Diodes) for emitting lights; a button configured to operate a plurality of operations, the plurality of operations comprises a colour change and a brightness change of the lights; a microcontroller configured to control the light at a pre-defined frequency range; and a power supply unit integrated within the body to provide power to the device.

In an embodiment of the present invention, in case, the button is pressed for long duration, the microcontroller changes the colour of the lights and in case, the button is single quick pressed, the microcontroller changes the brightness of the lights.

In the embodiment, the device comprises a plurality of LED's which are a plurality of RA95 LED's. In the embodiment, the device comprises a USB slot configured to power the microcontroller and the plurality of LED's. The embodiment of the present invention comprises at least one slot for hanging the device on an external support structure, a magnetic component for attaching the body to a metal surface or an aperture comprises a loop for hanging.

In the embodiment, the pre-defined frequency range of the device is 20 Hz to 40 Hz. In the embodiment the brightness of over 600 lumens comprising a plurality of color options including 3000 k, 6000 k, 660 nm, and 850 nm red lights is offered when the pre-defined frequency range is 20 Hz to 24 Hz.

The embodiment comprises a USB (Universal Serial Bus) port integrated with the body, to be utilized for establishing a wired connection between the device and an external power source via a voltage converter inbuilt within the USB in view of recharging the rechargeable battery.

While the invention has been described and shown with particular reference to the preferred embodiment, this will be apparent that variations might be possible that would fall within the scope of the present invention.

The preceding is a simplified summary to provide an understanding of some embodiments of the present invention. This summary is neither an extensive nor exhaustive overview of the present invention and its various embodiments. The summary presents selected concepts of the embodiments of the present invention in a simplified form as an introduction to the more detailed description presented below.

To further clarify advantages and features of the present invention, a more particular description of the invention will be rendered by reference to specific embodiment thereof, which is illustrated in the appended drawings. It is appreciated that these drawings depict only typical embodiments of the invention and are therefore not to be considered limiting of its scope. As will be appreciated, other embodiments of the present invention are possible utilizing, alone or in combination, one or more of the features set forth above or described in detail below.

### BRIEF DESCRIPTION OF THE DRAWINGS

These and other features, aspects, and advantages of the present invention will become better understood with regards to the following description, appended claims, and accompanying drawings where:

FIG. 1 illustrates a perspective view of a portable brain entertainment device 100 in accordance with various embodiments of the present invention;

FIG. 2 illustrates a top view of the portable brain entertainment device 100 in accordance with an exemplary embodiment of the present invention;

FIG. 3 illustrates a perspective view of the portable brain entertainment device 100 in accordance with another embodiment of the present invention;

FIG. 4A illustrates a perspective view of a multi-functional light flicker device, in accordance with an exemplary embodiment of the present invention;

FIG. 4B illustrates a bottom view of the multi-functional light flicker device, in accordance with an exemplary embodiment of the present invention; and

FIG. 4C illustrates an inside view of the multi-functional light flicker device, in accordance with an exemplary embodiment of the present invention.

### DETAILED DESCRIPTION OF THE INVENTION

The following description includes the preferred best mode of one embodiment of the present invention. It will be clear from this description of the invention that the invention is not limited to these illustrated embodiments, but the invention also includes a variety of modifications and embodiments thereto. Therefore, the present description should be seen as illustrative and not limiting. While the invention is susceptible to various modifications and alternative constructions, it should be understood, that there is no intention to limit the invention to the specific form disclosed, but, on the contrary, the invention is to cover all modifications, alternative constructions, and equivalents falling within the scope of the invention as defined in the claims.

In any embodiment described herein, the open-ended terms “comprising,” “comprises,” and the like (which are synonymous with “including,” “having” and “characterized by”) may be replaced by the respective partially closed phrases “consisting essentially of,” consists essentially of,” and the like or the respective closed phrases “consisting of,” “consists of, the like.

As used herein, the singular forms “a”, “an”, and “the” designate both the singular and the plural, unless expressly stated to designate the singular only.

Unless otherwise defined, all technical and scientific terms used herein have the same meaning as commonly understood by one of ordinary skill in the art to which this invention belongs. The present invention disclosed herein is illustrative only and not intended to be limiting.

Embodiments of the present invention will be described below in detail with reference to the accompanying drawings.

The present invention relates to a multifrequency neurostimulation device for improving cognitive performance of brain capable of delivering specific audiovisual stimuli to induce desired cognitive states in a user. The multifrequency neurostimulation device includes a portable brain entrainment device and or light flicker device that provides a means to optimize cognitive performance and enhance mental well-being. In an embodiment the invention provides a portable brain entrainment device that provides a convenient and effective solution for inducing desired cognitive states through synchronized audio-visual stimuli. The present invention has a compact structure and user-friendly interface, which makes the device suitable for use in various settings, offering users a portable means of enhancing their well-being and cognitive performance. The present device disclosed herein is capable of delivering specific audiovisual stimuli to induce desired brainwave states.

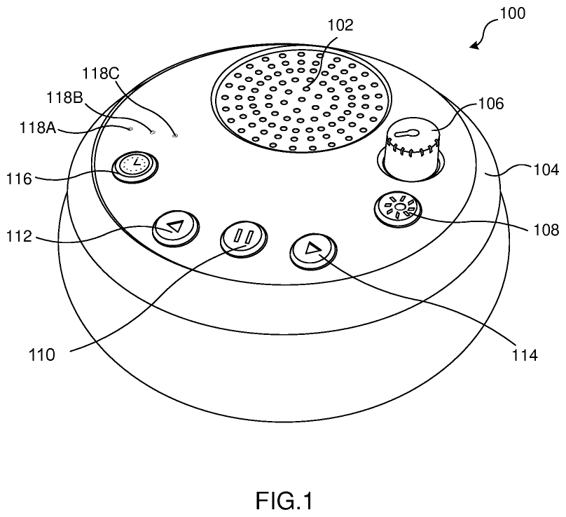

In reference to FIG. 1, the present invention, the portable brain entertainment device 100 comprises a body configured to encase a plurality of components, which includes a speaker 102, a light emitter 104, a volume adjustment knob 106, a light intensity adjustment knob 108, a play/pause button 110, a reverse button 112, a forward button 114, a timer 116, and one or more time indicating Light Emitting Diodes (LEDs) 118.

According to an embodiment of the present invention, the portable brain entrainment device is a multifrequency neurostimulation device for improving cognitive performance of brain comprises of a body that may have a top portion and bottom portion configured to house a plurality of components. The plurality of components comprises of a plurality of buttons for enabling a user to generate a command for plurality of operations. The plurality of operations includes but not limited to switching ON/OFF the device, play and pause, reverse and forward and color change or temperature.

In an embodiment of the present invention, the portable brain entrainment device discloses a sound module comprising the speaker 102 mounted on top of the body to generate a varying range of sound frequency, preferably an audible binaural beats and monotone sound frequencies. In reference to FIG. 1, the volume adjustment knob 106 may be configured to adjust volume of the speaker 102. The volume adjustment knob 106 allows the user to customize sound frequencies required for the desired cognitive state. The volume adjustment knob 106 allows for a more personalized and enjoyable listening experience, as a user can set the volume based on a personal comfort level or the comfort level of the environment the portable brain entertainment device is placed in. Further, the volume adjustment knob 106 allows adjusting volume to a lower level to prevent hearing damage caused by prolonged exposure to loud noise, thus avoiding hearing damages. Additionally, the volume adjustment knob 106 allows adapting the portable brain entertainment device 100 to various environments, such as a noisy or a quiet place by setting the volume accordingly thereby improving adaptability to different environments. In an additional embodiment of the present invention, the volume adjustment knob 106 may also work as a power ON and power OFF switch.

In an embodiment of the present invention, the binaural beats and the monotone sound may have a sawtooth frequency of 40 Hertz (Hz). It may be apparent to a person skilled in the art that the speaker 102 may be placed at any other location on the portable brain entertainment device 100 without departing from the scope of the invention.

In an embodiment of the present invention, the light emitter 104 may be placed around the top edge of the portable brain entertainment device 100 to flash a light. The light emitter 104 comprises a plurality of LEDs for emitting light or producing a flickering/flashing effect. The body is configured with the light intensity adjustment knob 108 for allowing the user to adjust the brightness of the light emitted from the light emitter 104. The user customizes light frequencies as per the requirement to achieve the desired cognitive state via the light intensity adjustment knob 108. Herein, the light emitter emits a plurality of color of light to enhance ergonomic usage of the device in day and evening without hindering circadian sleep cycle. The light emitting module and the sound module produce a light flickering/flashing effect and an audible sound effect of 40 Hz frequency simultaneously to stimulate gamma brainwave.

Furthermore, the varying range of frequency offered by the light emitting module and sound module is a pre-defined frequency range that ranges from 20 Hz to 45 Hz. In an embodiment of the present invention, the device is configured to provide frequency 20 Hz, 40 Hz, and 20-40 Hz in random order.

In an embodiment of the present invention, the light may have a frequency of 40 Hertz (Hz). Further, the light emitter 104 may be a Light Emitting Diode (LED) having a 50,000 lifespan hour that may last long. It may be apparent to a person skilled in the art that the light 104 may be placed at any other location on the portable brain entertainment device 100 without departing from the scope of the invention.

In one of the embodiments, the light emitter comprises a plurality of LED's to provide flicker effect frequency range is 20 Hz to 24 Hz offering high brightness of over 600 lumens comprising a plurality of color options including 3000 k, 6000 k, 660 nm, and 850 nm red lights.

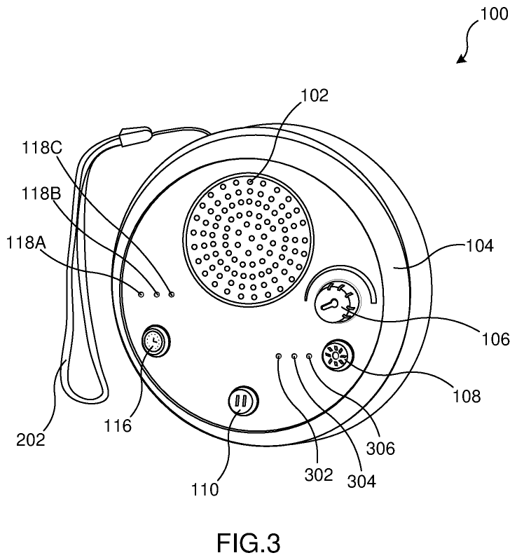

In an embodiment of the present invention, the light intensity adjustment knob 108 may be configured to adjust brightness of the light from the light emitter 104. The light intensity adjustment knob 108 allows a personalized and enjoyable lighting experience, as the user can set the brightness to a personal comfort level. Further, the light intensity adjustment knob 108 allows adjusting the brightness of the light to a lower level to save energy and extend the life of the light emitter, thus being energy efficient. Furthermore, the light intensity adjustment knob 108 allows adapting the light to different environments, activities and moods such as a working, reading, or a relaxing environment, thereby improving adaptability to different environments. In an illustrated embodiment of the present invention, as shown in FIG. 3, the set brightness may be visible to the user via one or more LED indicators, such as 25% by 302, 50% by 304, and 100% by 306.

In an embodiment of the present invention, the play/pause button 110, the reverse button 112 and the forward button 114 may be used to control the therapy session via play/pause, reverse, and forward application of the speakers 102 and the light emitter 104.

In an embodiment of the present invention, the timer 116 may be configured to effectively managing and tracking the amount of time a user need on a task to improve productivity and time efficiency, thus allowing a better time management. Further, the timer 116 also serves as an accounting tool helping the user to stay on a task and meet deadlines by clearly identifying how much time is remaining for a given task. Furthermore, the timer 116 provides convenience allowing the user to easily set reminders or alarms without having to check the time manually and can be helpful especially in cases when the user is multitasking or working in a busy environment. The timer 116 eliminates the need for an additional device to keep track of the time and thus the user need not purchase a plug-in timer or battery powered timer or any external device to manage light treatment.

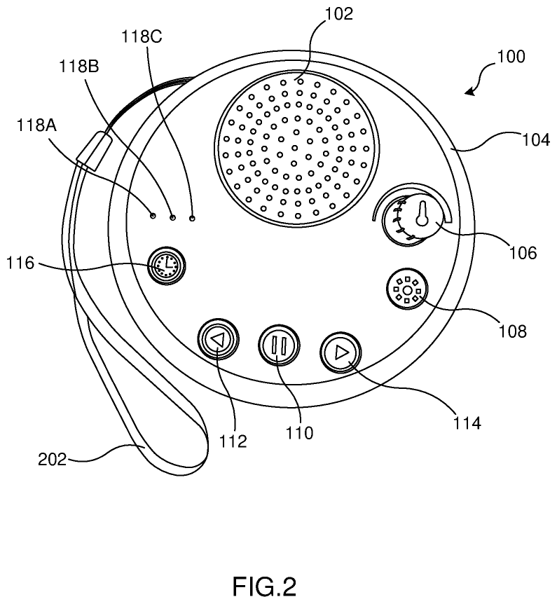

In an embodiment of the present invention, the set time may be indicated via one or more time indicating Light Emitting Diodes (LEDs) 118A, 118B, and 118C. In an illustrated embodiment of the present invention, as shown in FIG. 2, the one or more time indicating LEDs may represent set time, such as 15 mins by 118A, 30 mins by 118B, and 60 mins by 118C. In another embodiment of the present invention, as shown in FIG. 3, the one or more time indicating LEDs may represent set time, such as 1 hour by 118A, 2 hour by 118B, and 3 hour by 118C.

In an embodiment of the present invention, the light emitting module is configured to a customizable lighting experience to a variety of uses via an ability to change the color and temperature of light. Herein, the device comprises white, red and yellow colour LED having frequency of 20 Hz, 30 Hz, 40 Hz respectively. With the ability to adjust the light's color and temperature for a more tailored and satisfying experience, the light emitter 104 can be set to provide a customizable lightning experience, increased functionality, increased energy efficiency, and a variety of uses. These adjustment may be based on the desired ambiance, mood, activities (like reading, working, or relaxing), purposes (like home décor, photography, or therapeutic uses), and increased energy efficiency (like using a smart device to control the light's color and temperature to reduce energy consumption by using the necessary light for the tasks at hand).

The emission of light and sound frequency within 20-45 Hz range for targeted modulation of beta and gamma brainwaves. A unique approach for brainwave stimulation may be demonstrated by a triple-frequency mode for stimulation emission of 20 Hz, 30 Hz, and 40 Hz frequencies. The 20 Hz frequency adjustment enhances beta wave activity by an average of 43%. The 20 Hz frequency adjustment enhances beta wave activity by a maximum of 1200%. The 40 Hz frequency adjustment enhances gamma wave activity by an average of 47%. The 40 Hz frequency adjustment enhances beta wave activity by a maximum of 900%. The combination 20+40 Hz offers a synergetic effect, amplifying beta and gamma activity by over 45%.

In an embodiment of the present invention, the portable brain entertainment device 100 is lightweight and include a string 202, as shown in FIGS. 2 and 3, it may be easily carried from one place to another making it more convenient for on-the-go use. Also, because of the lightweight it may be easy to use and may cause less strain on muscles and joints. Further, the lightweight of the portable brain entertainment device 100 also allows it to be carried easily without causing fatigue while holding and carrying for extended periods of time making it more comfortable to use for longer durations.

In an embodiment of the present invention, the portable brain entertainment device 100 may include a rechargeable battery allowing mobility and no plugging into a power source. Thus, the portable brain entertainment device 100 may be adaptable and handy for usage when on the go as well as the user is free to use it wherever the user wants, such as home, workplace or even outside. Accordingly, the portable brain entertainment device is versatile and can even be used in emergency situations. The battery-powered nature of the portable brain entertainment device 100 also allows it to be used during power outrages or other disruptions to an electrical system providing a dependable supply of power when the user need it the most while reducing the amount of reliance that the user has on the electrical grid.

In an embodiment of the present invention, the portable brain entertainment device 100 is a Universal Serial Bus (USB) powered device with a built-in voltage converter making it more portable than a plug-in device as it does not require a separate power adapter or converter, allowing the user to carry it easily wherever the user go. The USB powered nature of the portable brain entertainment device 100 also eliminates the requirement of a power outlet or a cord making it more convenient to be used in situations where a power source is not readily available and reducing dependency on the power outlet. Thus, the USB powered portable brain entertainment device 100 with built-in converters allow the user to use the device in any location whether it is in home, office or outdoors without being restricted by the availability of the power outlet.

Thus, the portable brain entertainment device 100 encompasses numerous advantages. Since the portable brain entertainment device 100 can produce flickering/flashing effect of 40 Hz frequency and audible sound 40 Hz sawtooth frequency simultaneously, it maximizes the ability of the brain to produce more gamma brain waves for plethora of health benefits. Further, since the portable brain entertainment device 100 is portable, it can be used in the car, bathroom, office, on vacation, on a plane, camping outdoor or the like making it versatile for go-to use. Further, since the portable brain entertainment device produces variety of color and temperature, it can benefit the user by being used in the morning, afternoon and night without affecting the sleep patterns. Furthermore, since the portable brain entertainment device 100 is battery powered, it is mobile, power source independent and cost-effective due to no additional costs such as energy or power adapters. Additionally, since the portable brain entertainment device is USB powered with built-in converter, it is highly convenient, portable and mobile by allowing the user to use it in any region of the world without a need to purchase an additional AC voltage converter. Additionally, since the user may take the portable brain entertainment device 100 anywhere while on the move, the portable brain entertainment device 100 increases productivity of users, such as business traveller, freelancers, and professional who work remotely.

It has thus been seen that the portable brain entertainment device according to the present invention achieves the purposes highlighted earlier. The portable brain entertainment device can in any case undergo numerous modifications and variants, all of which are covered by the same innovative concept; moreover, all of the details can be replaced by technically equivalent elements. In practice, the materials used, as well as the shapes and sizes, can be whatever according to the technical requirements.

The present invention relates to a multi-functional light-flickering device that is portable and multi-functional, capable of producing light flicker at a frequency range of 20 Hz to 24 Hz, offering high brightness of over 600 lumens, providing multiple color options with 3000 k, 6000 k, 660 nm, and 850 nm red lights, as well as having a powerful power bank exceeding 10,000 mAh, among other features.

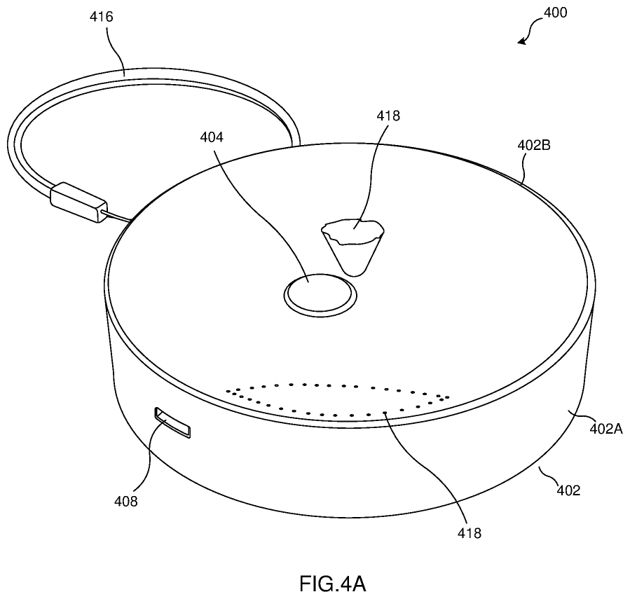

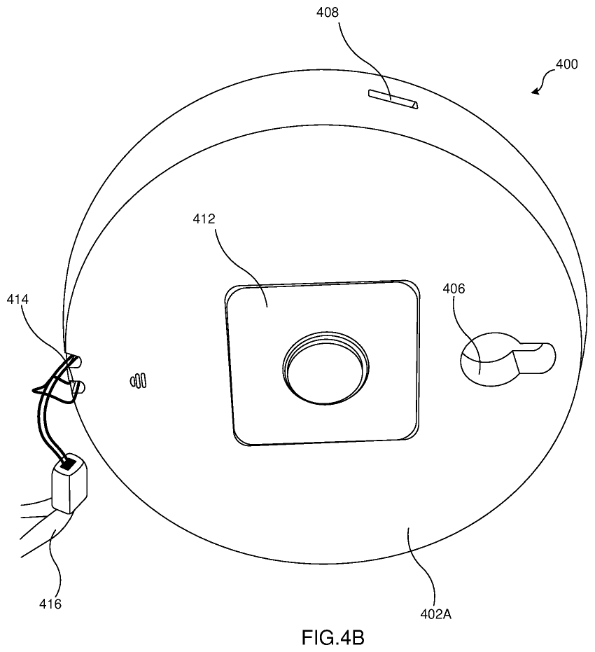

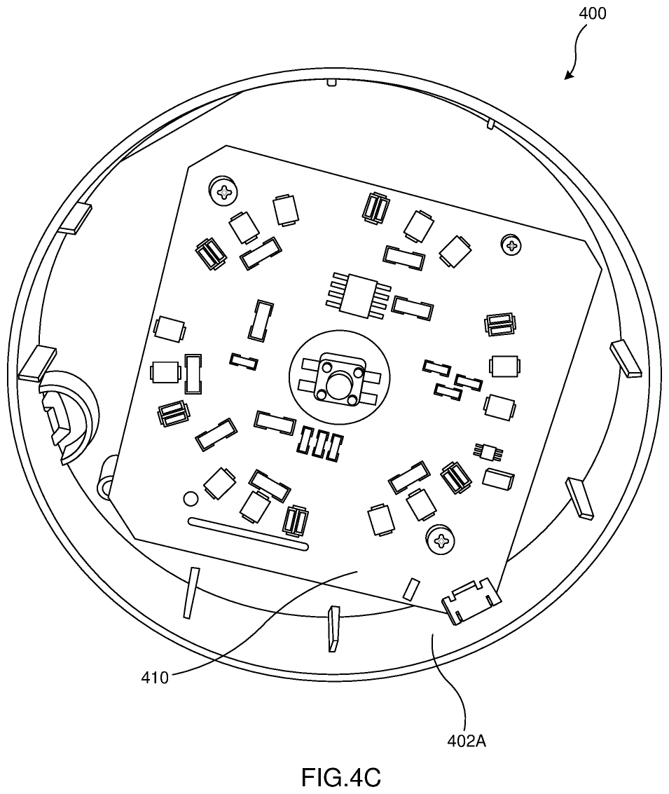

Referring now to FIGS. 4A, 4B and 4C to describe various exemplary embodiments of the present disclosure. FIG. 4A illustrates a perspective view of a multi-functional light flicker device 400, in accordance with an exemplary embodiment of the present disclosure.

Referring to FIGS. 4A, 4B and 4C, the multi-functional light flicker device 400 for stimulating beta brain waves comprises a body 402, a button 404, at least one slot 406, a USB slot 408, a microcontroller 410, a magnetic component 412, apertures 414, a loop 416, a power source, a plurality of lights, and so forth.

The multi-functional light flicker device 400 may comprise the body 402 having a bottom portion 402a and a top portion 402b. The body 402 may be made of plastic, or any other materials. The body 402 may be configured to hold a plurality of components of the light flicker device 400.

The bottom portion 402a may be detachable from the top portion 402b of the body 402. In an embodiment, the body 402 may be a circular shaped body. In another embodiment, the body 402 may be of any definite shape.

The bottom portion 402a of the body 402 may comprise the apertures 414. Moreover, a loop 416 may be inserted into the apertures 414 present at the bottom portion 402a of the body 402, as seen in FIG. 4B.

The multi-functional light flicker device 400 may comprise the button 404 configured to operate a plurality of operations. The button 404 may be located at the center of the top portion 402b of the body 402.

According to embodiments of the present disclosure, the plurality of operations may comprise a colour change, a brightness change, and so forth.

In one embodiment, the button 404 when pressed for a longer than a predetermined time, the microcontroller 410 changes the colour of the plurality of lights. In one embodiment, the button 404 when pressed once for duration shorter than a predetermined time, the microcontroller 410 changes the brightness of the plurality of lights.

In an embodiment, the button 404 may be a toggle button. The brightness may be high brightness of over 600 lumens.

Referring to FIG. 4C, the microcontroller may be placed inside the bottom portion 202a of the body.

The multi-functional light flicker device 400 may comprise at least one slot 406 for hanging the multi-functional light flicker device 400 on an external support structure.

The power source may be configured to provide power for greater flexibility and portability for any location. The power source may be a battery, a power bank with a capacity of over 10,000 mAh, and so forth.

The multi-functional light flicker device 400 may comprise the plurality of lights. In one embodiment, the plurality of lights may be light emitting diodes (LEDs). The plurality of lights may be a plurality of RA95 LED's. The plurality of lights may have multiple color options with 3000k and 6000k and 660 nm and 850 nm red lights. The plurality of lights may be used as a camping light to keep travellers alert. The plurality of LED's of the light flicker is configured to stimulate beta brainwave at a range of 20 Hz to 24 Hz.

The top portion 402b of the body 402 may have holes 418 of any definite shape through which the light emits out of the body 402, as seen in FIG. 4A.

The USB slot 408 may be configured to power the microcontroller 410 and the plurality of lights via external means. The multipurpose light flicker device 400 may be USB powered by the USB slot 408, making it portable and usable anywhere.

The microcontroller 410 may be configured to control the multi-functional lighting flicker device 400 at a predefined frequency range. According to embodiments of the present disclosure, the predefined frequency range may range in 20 Hz to 24 Hz. Further, the microcontroller 410 may further comprise means for controlling the colour of the plurality of lights.

The magnetic component 412 may be configured to attach the multi-functional light flicker device 400 to an external support structure. Further, the external support structure may be made of a metal surface complementary to the magnetic component.

In one embodiment, the magnetic component 412 may be attached at a base of the bottom portion 402a of the body 402. The magnetic component 412 may feature a magnetic attribute for use in gyms.

The multi-functional light flicker device 400 may comprise an attachment with a capability to stand on its own or be hung.

According to an embodiment of the present disclosure, the multi-functional light flicker device 400 may produce 20-24 Hz light flicker to stimulate beta brain waves, much like how 40 Hz light flicker may stimulate gamma brain waves.

The light flicker device 400 produces 20-24 Hz light flicker help a user to stay on task with focus.

In an exemplary embodiment, the light flicker device 400 is a small, battery-operated, portable device that may be positioned close to a workspace. The light flicker device 400 emits a minute light flicker that is hardly perceptible but has a significant impact on the brain.

Without the need for caffeine or other stimulants, the user may feel more focused and alert after using the light flicker device 400 for just a few minutes. Many more people who experience ADHD symptoms or difficulty focusing on tasks may benefit from the light flicker device 400. The light flicker device 400 is non-invasive, safe, and has no negative side effects.

Without the use of stimulants, the light flicker device 400 offers a safe and efficient method to stimulate beta brain waves.

The present invention relates to the light flicker device 400 that utilizes light frequency to induce greater beta brain wave activity in the brain. The light flicker device 400 is battery-powered and portable, making it easily accessible and user-friendly for individuals seeking to improve their cognitive performance.

The light flicker device 400 of the present invention is small, adaptable, and usable in a wide range of contexts, making it simple for users to incorporate it into their daily routines.

Additionally, the light flicker device's 400 offers portability, which makes it a desirable choice for users looking to enhance their cognitive function while on the go.

The invention is particularly designed for use while working, reading, exercising, camping, or traveling, and offers several advantages, such as providing high brightness and multiple color options to cater to various needs. The power bank function allows the invention to be used as a charger for electronic devices, making it versatile and practical. The lightweight and small size makes it portable and easy to carry anywhere. The magnetic feature makes it convenient to use in gyms or other settings where metal surfaces are available. The light can be placed in the background or in the peripheral vision in order to stimulate and entrain the brain.

New characteristics and advantages of the invention covered by this document have been set forth in the foregoing description. It is to be expressly understood, however, that the expressions used in foregoing are for the purpose of illustration only and are not intended as a definition of the limits of the invention. Changes in the specification are capable of being made in detail without exceeding the scope of the invention by those who are skilled in the art. The scope of the invention is described in the appended claims hereinafter.

What is claimed is:

1. A multifrequency neurostimulation device for improving cognitive performance of brain, the device comprises of:
a body having a top portion and bottom portion configured to house a plurality of components, the components comprising:
a plurality of buttons for enabling a user to generate a command for plurality of operations;
a light emitting module configured to emit light frequency of a varying range, wherein a light adjustment knob is configured for allowing the user to customize light frequencies required for a desired cognitive state;
a sound module configured to generate a varying range of sound frequency, wherein a volume adjustment knob is configured for allowing the user to customize sound frequencies required for the desired cognitive state;
a microcontroller to control the operation of the light emitting module and the sound module;
a timer configured for tracking time duration of emitted lights and/or sound, wherein a plurality of time-indicating LED (Light emitting diodes) lights indicates remaining time duration; and
a rechargeable battery configured on the body for supplying power to electrical and electronic component associated with the device.

2. The device as claimed in claim 1, wherein the varying range of frequency is a pre-defined frequency range, the pre-defined frequency range is 20 Hz to 45 Hz.

3. The device as claimed in claim 1, wherein the device is configured to provide frequency 20 Hz, 40 Hz, and 20 to 40 Hz in random order.

4. The device as claimed in claim 1, wherein the light emitting module is configured to include a plurality of LED's to produce light flicker effect for stimulating beta brain waves and light emitters for gamma brainwaves.

5. The device as claimed in claim 4, wherein the plurality of LED's of the light flicker is configured to stimulate beta brainwave at a range of 20 Hz to 24 Hz.

6. The device as claimed in claim 5, wherein the beta brainwave at the range of 20 Hz to 24 Hz offers high brightness of over 600 lumens comprising a plurality of color options including 3000 k, 6000 k, 660 nm, and 850 nm red lights.

7. The device as claimed in claim 1, wherein the light emitter module and the sound module produce a light flickering/flashing effect and an audible sound effect of 40 Hz frequency simultaneously to stimulate gamma brainwave.

8. The device as claimed in claim 1, wherein the plurality of operations includes but not limited to switching ON/OFF the device, play, pause, reverse, forward and color change or temperature.

9. The device as claimed in claim 1, wherein a USB (Universal Serial Bus) port integrated with the body, to be utilized for establishing a wired connection between the device and an external power source via a voltage converter inbuilt within the USB in view of recharging the rechargeable battery.

10. The device as claimed in claim 1, wherein the device comprises white, red and yellow colour LED having frequency of 20 Hz, 30 Hz, 40 Hz respectively.

11. The device as claimed in claim 1, wherein the light emitting module is configured to a customizable lighting experience to a variety of uses via an ability to change the color and temperature of light.

12. A portable brain entrainment device for stimulating gamma brain wave patterns to improve brain health, the device comprises of:
a body configured to encase a plurality of components, the components includes:
a speaker to generate binaural beats and monotone sound frequencies;
a light emitter comprising a plurality of LED's;
a volume adjustment knob configured to adjust the volume of the speaker;
a light intensity adjustment knob configured to adjust the brightness of the light from the light emitter;
a microcontroller to control the operation of the light emitter and the speaker;
a timer configured for effectively managing and tracking amount of time a user needs on a task to improve productivity and time efficiency;
a power supply unit to supply power to the plurality of components; and
a USB port with built-in converters allowing the user to connect an external power source to recharge a power supply unit;
wherein the speaker and the light emitter produce an audible sound and light flickering/flashing effect of 40 Hz frequency simultaneously;
wherein the light emitter is configured to a customizable lighting experience to a variety of uses via an ability to change the color and temperature.

13. The device as claimed in claim 12, wherein the light emitter emits a plurality of color of light to enhance ergonomic usage of the device in day and evening without hindering circadian sleep cycle.

14. The device as claimed in claim 12, wherein the light emitter has a 50,000 lifespan and a full spectrum of at least RA90.

15. The device as claimed in claim 12, wherein the light intensity adjustment knob allows adjustment of brightness of light at varying percentage such as 25%, 50%, and 100%.

16. The device as claimed in claim 12, wherein the power supply unit comprises a rechargeable battery to provide mobility to the device.

17. A portable multi-functional light flicker device for stimulating beta brain waves to improve cognitive performance of the brain, the device comprises of:
a body having a top portion and bottom portion configured to hold a plurality of components of the device;
a plurality of LEDs (Light Emitting Diodes) for emitting lights;
a button configured to operate a plurality of operations, the plurality of operations comprises a colour change and a brightness change of the lights;
a microcontroller configured to control the light at a pre-defined frequency range; and
a power supply unit integrated within the body to provide power to the device;
wherein the pre-defined frequency range is 20 Hz to 24 Hz offering high brightness of over 600 lumens comprising a plurality of color options including 3000 k, 6000 k, 660 nm, and 850 nm red lights;
wherein when the button is long pressed, the microcontroller changes the colour of the lights and when the button is single quick pressed, the microcontroller changes the brightness of the lights.

18. The device as claimed in claim 17, wherein the plurality of LED's are a plurality of RA95 LED's.

19. The device as claimed in claim 17, wherein the device comprises a USB slot configured to power the microcontroller and the plurality of LED's.

20. The device as claimed in claim 17, wherein the device comprises at least one or combination of a slot for hanging the device on an external support structure, a magnetic component for attaching the device a metal surface, an aperture to tie a loop for hanging.
